(19) United States
(12) Patent Application Publication
(10) Pub. No.: US 2008/0069887 A1
(43) Pub. Date: Mar. 20, 2008

(54) Method for nanoparticle surface modification

(75) Inventors: Jimmie R. Baran, Prescott, WI (US); Duane D. Fansler, Dresser, WI (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/522,075

(22) Filed: Sep. 15, 2006

(51) Int. Cl. A61K 9/50

(52) U.S. Cl. 424/490; 264/4.1; 977/906

(57) ABSTRACT

The present disclosure discloses a method for making surface-modified nanoparticles. The surface of a nanoparticle is modified with an aminorganosilane and an alkylating agent in a one-pot synthesis to provide alkylamine surface-modified nanoparticles.

### FIELD

The present disclosure relates to a method for modifying the surface of a nanoparticle.

### BACKGROUND

Nanotechnology is the creation and utilization of materials, devices and systems through the control of matter on a nanometer scale to understanding new molecular organization and phenomena. The control of matter on the nanoscale plays an important role in many science and engineering fields today.

Modification of inorganic and organic nanoparticles promotes usefulness in a number of applications. The average diameter of the nanoparticles provides for greater surface area and functionality.

Synthetic routes can be used for the surface modification of nanoparticles. The surfaces of the particles may have functionality present due to surface oxidation, or intentional modification to facilitate handling and transportation requirements. Further, nanoparticles may be dispersed in solvents, and subsequently reacted with selected reagents to afford new functionalities, either protected or unprotected. Multi-step methods to modify particles for composite and polymer applications are described in U.S. Pat. No. 6,986,943 to Cook et.al.

A variety of methods are available for modifying the surface of nanoparticles including, e.g., adding a surface modifying agent to nanoparticles (e.g., in the form of a powder or a colloidal dispersion) and allowing the surface modifying agent to react with the nanoparticles. Surface modified inorganic particles, such as zirconia nanoparticles, include organic acids, for example, oleic acid and acrylic acid adsorbed onto the surface of the particle. Surface modified silica nanoparticles may be modified with silane modifying agents. Other surface modification processes are described in, e.g., U.S. Pat. No. 6,586,483 (Kolb et al.), U.S. Pat. No. 2,801,185 (Iler), and U.S. Pat. No. 4,522,958 (Das et al.), and herein incorporated by reference.

Multi-step nanoparticle modifications can decrease efficiency and adaptability of materials for future applications. Reagents for surface modification may be air sensitive, or hydrolytically unstable. Also, difficulty may occur in re-dispersing particles resulting in variable coverage of the particle surface. Products of multi-step reactions may be difficult to isolate, and redisperse in subsequent further steps reducing yields. Solvent incompatibility and micellularization of modified nanoparticles can further limit additional reaction, consistent surface modification, and usefulness of such materials.

Synthetic modification of nanoparticles can lead to physical property limitations. Attempts to dry, purify and isolate modified particles can lead to agglomerated or aggregated materials with poor dispersibility in solvents. Aggregation of materials during surface modification may result in nanoparticles which are difficult to redisperse, resulting in settling, and nonuniform surface functionalization.

### SUMMARY

The present disclosure is directed to a method of making surface-modified nanoparticles. A reaction mixture is provided, which comprises a nanoparticle component, at least one aminoorganosilane, at least one alkylating agent, and a solvent. The mixture is then agitated and sufficiently heated to form alkylamine surface-modified nanoparticles.

In another aspect of this disclosure, the surface-modified nanoparticles further comprise quaternary amine groups. The one step modification of nanoparticles provides for alkylamine and quaternary amine groups in a monolayer coverage on the nanoparticle surface.

In another aspect of this disclosure, the surface modification of nanoparticles is performed in a single vessel or one-pot synthesis, without additional separation and isolation steps found in multistep syntheses. The aminosilane functionalization of the nanoparticle, and the alkylation of the terminal aminosilane groups occur with sufficient heating and agitation.

In another aspect of this disclosure, the surface modified nanoparticles are essentially free of agglomeration in a solvent or combination of solvents. Further, the surface-modified nanoparticles can be dried, and then re-dispersed in solvents, wherein the nanoparticles are essentially free of agglomeration.

The one pot synthesis for the surface modification of nanoparticles with alkylamine functionality provides for efficient processing and adaptability. Further, this method provides for the formation of quaternary amine surface-modified nanoparticles. This approach allows for two reactions to occur in a one-pot synthesis: 1) surface modification of silica nanoparticles with an aminosilane surface modifying agent, and 2) alkylation and quaternization of terminal amine groups to occur in a one pot synthesis. This method, performed in solvent(s), including aqueous and mixed solvents, overcomes potential solubility and reactivity issues in relation to a two pot synthesis. A one pot synthesis provides for a more uniform surface modification of the nanoparticles with a statistical distribution of primary, secondary, tertiary, and quaternary amine groups present on the particle surface, as a function of the starting aminoorganosilane. The method provides for a reduction of processing steps. Nanoparticle agglomeration from purification and drying steps, along with solvent incompatibility may also be reduced. The formation of quaternary amine groups reduces the handling of quaternary amine salts separately, which are susceptible to hydrolysis.

The above summary of the present disclosure is not intended to describe each disclosed embodiment or every implementation of the present disclosure. The figures and the detailed description which follow, more particularly exemplify illustrative embodiments.

### DETAILED DESCRIPTION

For the following defined terms, these definitions shall be applied, unless a different definition is given in the claims or elsewhere in the specification The term “alkylamine” is defined as an analog of ammonia (NH₃), in which either one, two, or three hydrogen atoms of ammonia are replaced by organic radicals. General formulas are: (1) primary amines, —N(R¹R²), where R¹ and R² are both H; (2) secondary amines, —N(R¹R³), where R¹ is H and R³ is an alkyl group; and (3) tertiary amines, —N(R³)₂, where R³ is an alkyl group. The alkyl group attachment is merely a representative example of one group that may be attached to the N (nitrogen) of the amine groups.

The term “quaternary amine” is defined as —N(R³)₃⁺Z⁻, where N is cationic, Z represents an anion or counterion to the cationic N, and each R³ is an alkyl group. The alkyl group attachment is merely a representative example of one group that may be attached to the N of the amine groups. The amine group is functionalized so as to form an ionic species.

The term “nanoparticle” as used herein (unless an individual context specifically implies otherwise) will generally refer to particles, groups of particles, particulate molecules (i.e., small individual groups or loosely associated groups of molecules) and groups of particulate molecules that while potentially varied in specific geometric shape have an effective, or average, diameter that can be measured on a nanoscale (i.e., less than about 100 nanometers).

The term, “one-pot synthesis” is a method to improve the efficiency of a chemical reaction, whereby a reactant or reactants is subjected to successive chemical reactions in just one reactor. This strategy avoids an extended separation process and purification of the intermediate chemical compounds, saving both time and resources while increasing the chemical yield.

The terms “particle diameter” and “particle size” are defined as the maximum cross-sectional dimension of a particle. If the particle is present in the form of an aggregate, the terms, “particle diameter” and “particle size” refer to the maximum cross-sectional dimension of the aggregate.

The term “surface-modified nanoparticle” is defined as a particle that includes surface groups attached to the surface of the particle. The surface groups modify the character of the particle sufficient to form a monolayer, desirably a continuous monolayer, on the surface of the nanoparticle.

The recitation of numerical ranges by endpoints includes all numbers subsumed within that range (e.g., 1 to 5 includes 1, 1.5, 2, 2.75, 3, 3.80, 4, and 5).

As included in this specification and the appended claims, the singular forms “a”, “an”, and “the” include plural referents unless the content clearly dictates otherwise. Thus, for example, reference to a composition containing “a compound” includes a mixture of two or more compounds. As used in this specification and appended claims, the term “or” is generally employed in its sense including “and/or” unless the content clearly dictates otherwise.

Unless otherwise indicated, all numbers expressing quantities or ingredients, measurement of properties and so forth used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings of the present disclosure. At the very least, and not as an attempt to limit the application of the doctrine of equivalents to the scope of the claims, each numerical parameter should at least be construed in light of the number of reported significant digits and by applying ordinary rounding techniques. Not withstanding that the numerical ranges and parameters setting forth the broad scope of the disclosure are approximations, their numerical values set forth in the specific examples are reported as precisely as possible. Any numerical value, however, inherently contains errors necessarily resulting from the standard deviations found in their respective testing measurement.

The method of this disclosure describes making surface modified nanoparticle in a one-pot synthesis. This method also provides a means to perform two reactions in a single dispersion reducing solvent incompatibilities and inconsistent nanoparticle functionalization. Further, this method provides for the reactants of the mixture to be subjected to multiple chemical reactions without additional transfer of intermediates to separate vessels, further reducing the number of processing steps. Efficiency is increased as a result of a simple separation and purification process saving both time and resources with increases in chemical yield.

A method of this disclosure is further described, wherein a mixture comprises a nanoparticle component, at least one aminoorganosilane, at least one alkylating agent, and a solvent. The mixture is agitated with sufficient heating to form alkylamine surface-modified nanoparticles. The surface-modified nanoparticles further comprise primary, secondary, tertiary, and quaternary amine groups. The surface modified nanoparticles made of this method are essentially free of aggregation. Further, the surface-modified nanoparticles can be dried, readily dispersed in solvent, essentially free of aggregation.

The nanoparticles of the reaction mixture are inorganic. Suitable inorganic nanoparticles include silica and metal oxide nanoparticles including zirconia, titania, ceria, alumina, iron oxide, vanadia, antimony oxide, tin oxide, alumina/silica, iron oxide/titania, titania/zinc oxide, zirconia/silica, calcium phosphate, nickel oxide, zinc oxide, calcium hydroxylapatite, and combinations thereof. In one aspect of the invention, the nanoparticles preferably have an average particle diameter less than 100 nm, preferably no greater than about 50 nm, more preferably from about 3 nm to about 50 nm, even more preferably from about 3 nm to about 20 nm, most preferably from about 5 nm to about 10 nm. If the nanoparticles are aggregated, the maximum cross sectional dimension of the aggregated particle is within any of these preferable ranges.

Metal oxide colloidal dispersions include colloidal zirconium oxide, suitable examples of which are describe in U.S. Pat. No. 5,037,579 (Matchett). Further, colloidal titanium oxide examples may be fount in WO 00/06495 (Arney et. al.). Inorganic colloid dispersions are available from Nyacol Nano Technologies (Andover, Mass.).

In an exemplary embodiment, the unmodified silica particles may be used as the nanoparticle component of this disclosure. The nanoparticles may be in the form of a colloidal dispersion available under the produce designations NALCO 2326, 2327, 1130, 2359 (Nalco Chemical Company; Naperville, Ill.).

In another aspect, the nanoparticles are substantially individual, unassociated (i.e. non-aggregated), and dispersed without irreversible association. The term “associate with” or “associating with” includes, for example, covalent bonding, hydrogen bonding, electrostatic attraction, London forces, and hydrophobic interactions.

The nanoparticle component of this disclosure is surface-modified by the method described herein. The surface of the nanoparticle component may be modified with one or more amine surface modifying groups. A surface-modified nanoparticle is a particle that includes surface groups attached to the surface of the particle. The surface groups modify the hydrophobic or hydrophilic nature of the particle, including, but not limited to electrical, chemical, and/or physical properties. In some embodiments, the surface groups may render the nanoparticles more hydrophobic. In some embodiments, the surface groups may render the nanoparticles more hydrophilic. The surface groups may be selected to provide a statistically averaged, randomly surface-modified particle. In some embodiments, the surface groups are present in an amount sufficient to form a monolayer, preferably a continuous monolayer, on the surface of the particle.

In some situations where the nanoparticle is processed in solvent, the amine surface modifying groups may compatibilize the particle with the solvent for processing. In those situations, where the nanoparticles are not processed in solvent, the surface modifying group or moiety may be capable of preventing irreversible agglomeration of the nanoparticle.

In an exemplary embodiment of this disclosure, less than 80 percent of the available surface functional groups (e.g. Si—OH groups) of the nanoparticle are modified with a hydrophilic surface-modifying agent to retain hydrophilicity and dispersibility.

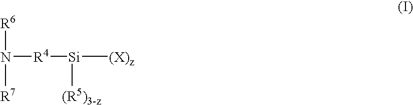

The aminoorganosilane as illustrated in formula (I) of this disclosure is referred to as a surface modifying agent. The surface modifying agent has at least two reactive functionalities. One of the reactive functionalities is capable of covalently bonding to the surface of the nanoparticles, and the second functionality is capable of being alkylated to form alkylamine groups. For example, if the nanoparticle is silica, the Si—OH groups of the nanoparticles are reactive with the X groups of the aminoorganosilane.

In one embodiment, for example, at least one X group is capable of reacting with the nanoparticle surface. In another aspect, the number of X groups ranges from 1 to 3, wherein further reaction of additional X groups may occur on the nanoparticle surface.

In an aspect of this disclosure, at least one aminoorganosilane, and more than one aminoorganosilane may be used for the surface modification, or in combination thereof.

The nanoparticle is surface-modified with aminoorganosilanes. The aminoorganosilane is of the formula (I). The aminoorganosilanes may comprise monoamine, diamine, and triamine functionality, wherein the amino groups may be within the chain or a terminal group. The aminoorganosilane is of the formula (I): wherein R⁶ and R⁷ are each independently hydrogen, linear or branched organic groups, alkyl groups having about 1 to about 16 carbon atoms (on average), aryl such as those selected from the group consisting of phenyl, thiophenyl, naphthyl, biphenyl, pyridyl, pyrimidinyl, pyrazyl, pyridazinyl, furyl, thienyl, pyrryl, quinolinyl, bipyridyl, and the like, alkaryl, such as tolyl, or aralkyl group, such as benzyl, and R⁶ and R⁷ may be attached by a cyclic ring, as represented by pyridine or pyrrole moiety; R⁴ is a divalent species, selected from linear or branched organic groups including alkyl having from 1 to 16 carbon atoms (on average), aryl, cycloalkyl, alkylether, alkylene (optionally including one or more caternary N (amine) groups in the chain or pendent, for example in formula (Ia) and combinations thereof;

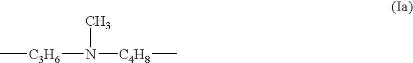

R⁵ is a independently selected from the group comprising alkyl, having from about 1 to about 16 carbon atoms (on average), aryl, and combinations thereof; X is a halide, alkoxy, acyloxy, hydroxyl and combinations thereof; and z is an integer from 1 to 3. Further, alkyl groups can be straight or branched chain, and alkyl and aryl groups can be substituted by noninterfering substituents that do not obstruct the functionality of the aminoorganosilane. The reaction mixture comprises at least one aminoorganosilane, but may comprise more than one aminoorganosilane, or combinations thereof.

The aminoorganosilane is used in amounts sufficient to react with 1 to 100% of the available functional groups on the inorganic nanoparticle (for example, the number of available hydroxyl functional groups on silica nanoparticles). The number of functional groups is experimentally determined where a quantity of nanoparticles is reacted with an excess of surface modifying agent so that all available reactive sites are functionalized with a surface modifying agent. Lower percentages of functionalization may then be calculated from the result. In an exemplary embodiment, the weight ratio of aminoorganosilane to nanoparticles ranges from 1.5:100 to 15:100.

The aminoorganosilanes are further selected from the group of aminoalkylsilanes, aminoarylsilanes, aminoalkoxysilanes, aminocycloalkylsilanes, and combinations thereof. The aminoorganosilane is present in the reaction mixture to functionalize at least 30 percent of the functional groups on the surface on the nanoparticle. Examples of aminoorganosilanes include 3-aminopropyltriethoxysilane, 3-aminopropyltrimethoxysilane, 4-aminobutyltriethoxysilane, m-aminophenyltrimethoxysilane, p-aminophenyltrimethoxysilane, aminophenyltrimethoxysilane, 3-aminopropyltris(methoxyethoxyethoxy)silane, 2-(4-pyridylethyl)triethoxysilane, 2-(trimethoxysilylethyl)pyridine, N-(3-trimethoxysilylpropyl)pyrrole, 3-(m-aminophenoxy)propyltrimethoxysilane, aminopropylsilanetriol, 3-aminopropylmethyldiethoxysilane, 3-aminopropyldiisopropylethoxysilane, 3-aminopropyldimethylethoxysilane, N-(2-aminoethyl)-3-aminopropyltrimethoxysilane, N-(6-aminohexyl)aminomethyltrimethoxysilane, N-(6-aminohexyl)aminopropyltrimethoxysilane, N-(2-aminoethyl)-11-aminoundecyltrimethoxysilane, (aminoethylaminomethyl)phenethyltrimethoxysilane, N-3-[(amino(polypropylenoxy)]aminopropyltrimethoxysilane, N-(2-aminoethyl)-3-aminopropylsilanetriol, N-(2-aminoethyl)-3-aminopropylmethyldimethoxysilane, N-(2-aminoethyl)-3-aminoisobutylmethyldimethoxysilane, (aminoethylamino)3-isobutyldimethylmethoxysilane, (3-trimethoxysilylpropyl)diethylenetriamine, n-butylaminopropyltrimethoxysilane, N-ethylaminoisoburyltrimethoxysilane, N-methylaminopropyltrimethoxysilane, N-phenylaminopropyltrimethoxysilane, 3-(N-allylamino)propyltrimethoxysilane, N-cyclohexylaminopropyltrimethoxysilane, N-phenylaminomethyltriethoxysilane, N-methylaminopropylmethyldimethoxysilane, bis(2-hydroxyethyl)-3-aminopropyltriethoxysilane, diethylaminomethyltriethoxysilane, (N,N-diethyl-3-aminopropyl)trimethoxysilane, 3-(N-N-dimethylaminopropyl)trimethoxysilane, and combinations thereof.

In an exemplary embodiment, 3-(N,N-dimethyl aminopropyl) trimethoxysilane may be used to modify the surface of the nanoparticles.

The alkylating agent reacts via a nucleophilic substitution reaction with the amino group of the aminoorganosilane coupled to the nanoparticle to form an alkylamines and quaternary ammonium salts. The alkylating agent is of the formula (II):

Y—R⁸-Z (II)

wherein Y may be hydrogen, fluorine, hydroxyl, allyl, vinyl ether or combinations thereof, or other groups which do not interfere with the alkylation of the amino group; R⁸ is a divalent species, selected from aliphatic (C₁ to C₂₄), cycloaliphatic, benzyl groups, alkylene (to include one or more caternary N (amine groups in the chain or pendent) or combinations thereof; and Z is a halide, tosylate, sulfate, functionalized sulfonates (e.g. 2-acrylamido-2-methyl-1-propanesulfonic acid), phosphate, hydroxyl group, or combinations thereof. The nucleophilic N of the aminoorganosilane attacks the electrophilic C of Y—R⁸-Z to displace Z. A new bond between N and the electrophilic C of Y—R⁸ is formed, thus forming the alkylated species of the quaternary amine group. The Z group, which is the leaving group of the alkylation reaction, forms the anion species of the quaternary ammonium salt as illustrated in formula (III).

Alkylation of amino groups with smaller alkyl halides generally proceeds from a primary amine to a quaternary amine. Selective alkylation may be accomplished by steric crowding on the amino group, which may reduce its nucleophilicity during alkylation. If the reacting amine is tertiary, a quaternary ammonium cation may result. Quaternary ammonium salts can be prepared by this route with diverse Y—R⁸ groups and many halide and pseudohalide anions.

In an exemplary embodiment, alkyl iodides, and alkyl bromides may be used to alkylate the aminoorganosilane.

In a further embodiment, the alkylating agent is an alkyl halide, for example, butyl bromide or lauryl chloride.

The amine group can be further alkylated to comprise a distribution of primary, secondary, tertiary, and quaternary amine groups forming a continuous monolayer coverage, or less than a monolayer of alkylamine and quaternary amine functionalization on the surface of the nanoparticle.

The quaternary amine of formula (III) is an ionic species, where Z⁻ is an anionic counterion to the cation, N⁺, of the quaternary ammonium group. The quaternary ammonium group is covalently bonded to the nanoparticle, ◯, at group X, where z=1-3. The reaction mixture contains at least one alkylating agent, but may also comprise more than one alkylating agent or combinations thereof.

It is understood that the X group attached to the silanes may further react with other silanes to form siloxanes, and/or react with other functional groups on the same or another nanoparticles. For example, formula (IIIa and IIIb) illustrate two plausible reactions of the X groups representing attachment. Other reactions with the X group may be considered.

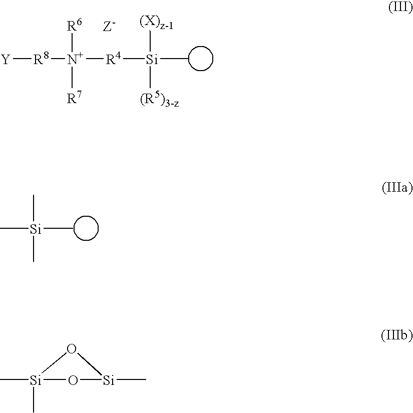

In an exemplary embodiment, the aminoorganosilane functionalized nanoparticles of this disclosure are further reacted with an alkylating agent. In a one-pot synthesis, the alkylating agent reacts react with the amino groups of the organosilane coupled to the nanoparticle.

In an exemplary embodiment, the alkyl halides react with the amines to form an alkyl-substituted amine followed by subsequent surface modification of the nanoparticles.

In an exemplary embodiment, the molar ratio of alkylating agent to aminoorganosilane ranges from 5:1 to 1:15. The amount of alkylating agent in the mixture is sufficient to quaternize the amino groups or alkylate at least a portion of the amino groups of the aminoorganosilane.

The surface-modified nanoparticles comprising alkylamine and quaternary amine groups are preferably individual, unassociated (non-aggregated) nanoparticles dispersed within the solvent or combination of solvents, where the nanoparticles do not irreversibly associate with each other. The surface-modified nanoparticles are dispersed within a solvent(s) such that the particles are free of particle agglomeration or aggregation.

The method of this disclosure further describes surface-modified nanoparticles comprising a monolayer of amine groups. The nanoparticle component may have surface modification or functionalization from a monolayer coverage to less than a monolayer coverage. The amine groups of the surface modification may comprise a distribution of primary, secondary, tertiary and quaternary amine groups. In a exemplary embodiment, the ratio of quaternary amine to tertiary amine groups ranges from 1:100 to 100:1 on the surface of the nanoparticle.

In an exemplary embodiment, the method of this disclosure can be further described wherein the surface functionalization of the nanoparticle is a continuous monolayer of alkylamine surface modified groups.

The reaction mixture of this disclosure contains a solvent or solvents for the dispersion of the nanoparticle component. Solvents useful for making surface-modified nanoparticles include water; alcohols selected from ethanol, propanol, methanol, 2-butoxy ethanol, 1-methoxy-2-propanol and combinations thereof; ketones selected from methyl ethyl ketone, methyl isobutyl ketone, acetone and combinations thereof; glycols selected from ethylene glycol, propylene glycol; dimethylformamide, dimethylsulfoxide, tetrahydrofuran, 1,4-dioxane, acetonitrile and combinations thereof. In the one-pot synthesis, polar solvents are used to disperse the unmodified nanoparticles and surface modified nanoparticles. The solvents in the one pot synthesis during surface modification of the nanoparticles disperse the particles. The alkylamine and/or quaternary amine surface groups of the nanoparticles provide for compatibility, such as solubility or miscibility.

In another embodiment of this disclosure, dried surface-modified nanoparticles are readily dispersible in solvent(s) and free of particle agglomeration and aggregation. The addition of solvents to dried surface-modified nanoparticles provides for a transparent mixture upon redispersion. Microscopy demonstrates individual particles dispersed within the solvent.

The hydrophilic surface groups, such as alkyl amines, covalently attached to a nanoparticle are re-dispersible in a solvent or in a combination of solvents. The dispersion of the surface-modified nanoparticles of this disclosure in a solvent ranges from 10 to 50 weight percent solids. In another aspect, the dispersion of the nanoparticles ranges from 15 to 40 weight percent solids. In a further aspect, the dispersion of the nanoparticles ranges from 15 to 25 weight percent solids.

Re-dispersed nanoparticles in solvents with reduced dispersibility yield hazy or cloudy solutions. Additionally, nanoparticles dispersed in a solvent with lower dispersibility can yield higher solution viscosities. The compatibility (e.g. miscibility) of dispersed surface modified particles in a solvent can be influenced factors such as the amount of surface modification on the nanoparticle, compatibility of the functional group on the nanoparticle with the solvent, steric crowding of the group on the particle, ionic interactions, and nanoparticle size, not to be all inclusive.

In another embodiment of this disclosure, the nanoparticles are surface modified with alkylamines, further comprising quaternary amine groups. Functionalization of the surface of the nanoparticle with an aminoorganosilane and alkylating the amino group to generate a quaternary amine group in a one-pot reaction can contribute to increased dispersibility in a solvent. Functionalization of the surface of the nanoparticle with a quaternary aminosilane, synthesized separately from the nanoparticle in a multi-step procedure contributes to lower dispersibility in a solvent. Reduced dispersibility of a nanoparticle from the multi-step procedure may be attributed to lower particle functionalization, steric crowding of functional groups, availability of functional groups from the silane to the nanoparticle, and the solubility of the quaternary aminosilane with the dispersed nanoparticle in a solvent. These factors or a combination of factors, not to be all inclusive, may be attributed to lower dispersibility.

The surface modified nanoparticles have surface amine groups that aid in the dispersion of the nanoparticle in solvents. The alkylamine and quaternary amine surface groups are present on the surface sufficient to provide nanoparticles that are capable of being dispersed without aggregation. The surface groups preferably are present in an amount sufficient to form a monolayer, preferably a continuous monolayer on the surface of the nanoparticle.

In one embodiment, the alkylamines and quaternary amines are represented by the formulas where e.g., —N(R⁶)₂ (primary); —N(R⁶R⁷) (secondary); —N(R⁷)₂ (tertiary); and —N((R⁷)₂YR⁸))⁺Z⁻ (quaternary), where R⁶ and R⁷ are each independently hydrogen, linear or branched organic groups, alkyl groups having about 1 to about 16 carbon atoms (on average), aryl such as those selected from the group consisting of phenyl, thiophenyl, naphthyl, biphenyl, pyridyl, pyrimidinyl, pyrazyl, pyridazinyl, furyl, thienyl, pyrryl, quinolinyl, bipyridyl, and the like, alkaryl, such as tolyl, or aralkyl group, such as benzyl, and R⁶ and R⁷ may be attached by a cyclic ring, as represented by pyridine or pyrrole moiety, R⁸ is a divalent species, selected from aliphatic (C₁ to C₂₄), cycloaliphatic, benzyl groups, alkylene (to include one or more caternary N (amine groups in the chain or pendent) or combinations thereof, Y can be hydrogen, fluorine, hydroxyl, allyl, vinyl ether, and combinations thereof, and Z is an ionic species from the alkylation reaction of the amine. The amine surface groups represent a distribution of amine group functionalities on the surface of nanoparticles.

In an exemplary embodiment, alcohols, water and combinations thereof are used as the solvent for making surface-modified nanoparticles.

In an exemplary embodiment, the mixture is agitated and heated at a temperature sufficient to ensure mixing and reaction of the mixture with the nanoparticles ranging from 1.5 to 28 hours. The unmodified nanoparticle component is dispersed in water. The aminoorganosilane, and an alkylating agent are added with a solvent to comprise the reaction mixture. After surface-modifying the nanoparticle component, the surface modified nanoparticles are analyzed for amine group composition.

Agitation of the reaction mixture can be obtained by shaking, stirring, vibration, ultrasound, and combinations thereof.

The temperature of modifying the surface of the nanoparticles is sufficient for the one pot synthesis (one-pot reaction) to occur. In one aspect, the reaction temperature ranges from 80° C. to 110° C.

In an exemplary embodiment of this disclosure, the surface-modified nanoparticles may be dried for 2 to 24 hours from 80° C. to 160° C. to remove solvent, water, and unreacted components. Solvent washing may be accomplished to further purify the nanoparticles of this disclosure.

Heating of the reaction mixture and drying the surface-modified nanoparticles can be obtained by thermal, microwave, electrical, and combinations thereof.

Objects and advantages of this disclosure are further illustrated by the following examples. The particular materials and amounts thereof, as well as other conditions and details, recited in these examples should not be used to unduly limit this disclosure.

### EXAMPLES

All solvents and reagents were obtained from Sigma-Aldrich Chemical Company, Milwaukee, Wis., unless otherwise noted. Nalco 2326 colloidal silica was obtained from Nalco Chemical Company (Bedford Park, Ill., USA). All percents and amounts are by weight unless otherwise specified.

Nuclear Magnetic Resonance spectroscopic analysis was carried out using a 400 MHz Varian NOVA solid-state spectrometer. (Palo Alto, Calif., USA). Samples were packed in 5 mm rotors. ¹⁵N and ¹³C CP/MAS were collected using a 5 mm MAS NMR probe. ¹⁵N spectra were referenced to liquid ammonia through a secondary reference of ¹⁵N labeled glycine. The quaternary peak at 55 ppm and the ternary peak at 45 ppm were used to determine the degree of quaternization.

Preparation of N-trimethoxysilylpropyl-N,N-dimethylbutylammoniumbromide: N,N-dimethylaminopropyltrimethoxysilane (10 g; Gelest, Inc., Morrisville, Pa., USA), and butyl bromide (9.89 g) in diethyl ether (50 g; Mallinckrodt Baker, Phillipsburg, N.J., USA) were placed in a suitable container, and stirred with a magnetic stir bar at room temperature for 48 hours. Diethyl ether was removed using a rotary evaporator to isolate 16.25 g of product. Analysis of the product by ¹⁵N NMR spectroscopy showed the amine quaternization to be 100 percent.

### Comparative Example 1

A mixture of Nalco 2326 colloidal silica (100 g), N-trimethoxysilylpropyl-N,N-dimethylbutylammoniumbromide (5.88 g) and 1-methoxy-2-propanol (117.5 g; Alfa Aesar, Ward Hill, Mass., USA) were mixed in a 3-neck round bottom flask equipped with a mechanical stirrer at 80° C. for 1 hour. The product was then isolated by drying in an oven at 130° C. (15.03 g). Solubility of the surface-modified nanoparticles yielded a transparent solution with less than 2 weight percent in water. At greater than 2 weight percent of the surface-modified nanoparticles, the solution was hazy with particulate matter settling. The solution viscosity increased significantly at greater than 2 weight percent surface-modified nanoparticles as compared to the transparent solution with less than 2 weight percent surface-modified nanoparticles.

### Example 1

A mixture of Nalco 2326 colloidal silica (100 g), N,N-dimethylaminopropyltrimethoxysilane (5.88 g), and 1-methoxy-2-propanol (117.5 g) were mixed in a three-neck round bottom flask equipped with a mechanical stirrer at 80° C. for 1 hour. Lauryl chloride (5.8 g)in 1-methoxy-2-propanol (20 g) was added to the mixture and stirred for an additional 18 hours at a temperature of 80° C. The surface-modified nanoparticles were isolated by drying in an oven at 130° C. (15.03 g). The surface-modified nanoparticles were soluble in water at greater than 20 weight percent yielding a transparent solution without an increase in solution viscosity. Quaternarization of the amine was greater than 20% based on ¹⁵N NMR spectroscopic analysis.

### Example 2

A mixture of Nalco 2326 colloidal silica (100 g), N,N-dimethylaminopropyltrimethoxysilane (5.88 g), and 1-methoxy-2-propanol (117.5 g) were mixed in a three-neck round bottom flask using a mechanical stirrer at 80° C. for 1 hour. Butyl bromide (3.88 g) in 1-methoxy-2-propanol (20 g) was added to the mixture and stirring was continued for an additional 18 hours while the reaction temperature was maintained at 80° C. The surface-modified nanoparticles were isolated by drying in an oven at 130° C. (22.3 g). The surface-modified were soluble in water at greater than 20 weight percent yielding a transparent solution without an increase in solution viscosity. The surface-modified nanoparticles were soluble in water at greater than 20 weight percent yielding a solution without an increase in solution viscosity. Quaternarization of the amine was greater than 20% based on ¹⁵N NMR spectroscopic analysis.

We claim:

1. A method for making surface-modified nanoparticles comprising:
a) providing a mixture comprising a nanoparticle component; at least one aminoorganosilane; at least one alkylating agent; and a solvent; and
b) agitating the mixture with sufficient heating to form alkylamine surface-modified nanoparticles.

2. The method of claim 1, further comprising quaternary amine surface modified nanoparticles.

3. The method of claim 1, further comprising the step of drying the surface-modified nanoparticles.

4. The method of claim 1, wherein the surface-modified nanoparticles are essentially free of agglomeration.

5. The method of claim 1, wherein the nanoparticle surface comprises a monolayer of primary amine, secondary amine, tertiary amine, and quaternary amine groups.

6. The method of claim 1, wherein the nanoparticle component is selected from the group consisting of silica, titania, alumina, nickel oxide, zirconia, vanadia, ceria, iron oxide, antimony oxide, tin oxide, zinc oxide, alumina/silica, iron oxide/titania, titania/zinc oxide, zirconia/silica, calcium phosphate, calcium hydroxyapatite and combinations thereof.

7. The method of claim 1, wherein the aminoorganosilane is selected from the groups consisting of aminoalkylsilanes, aminoarylsilanes, aminoalkoxysilanes, aminocycloalkylsilanes, and combinations thereof.

8. The method of claim 7, wherein the aminoorganosilane is of the formula: 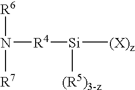
wherein R⁶ and R⁷ are each independently hydrogen, linear or branched organic groups; R⁴ is a divalent species, selected from linear or branched organic groups; R⁵ is independently selected from the group comprising alkyl, aryl, and combinations thereof; X is a halide, alkoxy, acyloxy, hydroxyl and combinations thereof; and z is an integer from 1 to 3.

9. The method of claim 7, wherein the aminoorganosilane is 3-(N,N-dimethyl aminopropyl)trimethoxysilane.

10. The method of claim 7, wherein the aminoorganosilane is 3-(N,N-diethylaminopropyl)trimethoxysilane.

11. The method of claim 1, wherein the alkylating agent has the formula:
Y—R⁸-Z
wherein Y is hydrogen, fluorine, hydroxyl, allyl, vinyl ether or combinations thereof; R⁸ is a divalent species, selected from aliphatic, cycloaliphatic, benzyl groups, or combinations thereof; and Z is a halide, tosylate, sulfate, functionalized sulfonate, phosphate, hydroxyl, or combinations thereof.

12. The method of claim 11, wherein the alkylating agent comprises C₁-C₂₄ halides.

13. The method of claim 1, wherein the solvent is selected from the group consisting of water, ethanol, propanol, methanol, and 1-methoxy-2-propanol, acetone, methyl ethyl ketone, methyl isobutyl ketone, ethylene glycol, propylene glycol, 2-butoxy ethanol, dimethylformamide, dimethylsulfoxide, tetrahydrofuran, 1,4-dioxane, acetonitrile and combinations thereof.

14. The method of claim 1, wherein the dispersion of the surface-modified nanoparticles ranges from 10 to 50 weight percent.

15. The method of claim 1, wherein the molar ratio of the alkylating agent to the aminoorganosilane ranges from 5:1 to 1:15.

16. The method of claim 1, wherein the surface-modified nanoparticle has a monolayer coverage to less than a monolayer coverage.

17. The method of claim 1, wherein the amount of aminoorganosilane is present in an amount sufficient to functionalize less than 80 percent of the functional groups on the surface of the nanoparticle.

18. The method of claim 1, wherein the amount of the alkylating agent is sufficient to quaternize the amino groups of the aminoorganosilane.

19. The method of claim 1, wherein the amount of alkylating agent is sufficient to alkylate at least a portion of the amino groups of the aminoorganosilane.

20. The method of claim 1, wherein the surface-modified nanoparticles comprise a mixture of —N(R⁶)₂ groups; —N(R⁶R⁷) groups; —N(R⁷)₂ groups; and —N((R⁷)₂YR₈))⁺ Z⁻ groups represented by the formula: 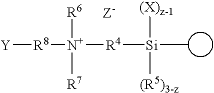
wherein R⁶ and R⁷ are each independently hydrogen, linear or branched organic groups and combinations thereof; R⁴ is a divalent species, selected from linear or branched organic groups and combinations thereof; R⁵ is independently selected from the group comprising alkyl, aryl, and combinations thereof; X is a halide, alkoxy, acyloxy, hydroxyl and combinations thereof; R⁸ is a divalent species, selected from aliphatic, cycloaliphatic, benzyl, alkylene and combinations thereof; Y is hydrogen, fluorine, hydroxyl, allyl, vinyl ether, and combinations thereof; Z is a halide, tosylate, sulfate, functionalized sulfonate, phosphate, hydroxyl, and combinations thereof; and z is an integer from 1 to 3; thereof on the surface of the nanoparticle.
